(19) United States
(12) Patent Application Publication
(10) Pub. No.: US 2008/0195176 A1
(43) Pub. Date: Aug. 14, 2008

(54) Raynaud's Conditions

(75) Inventors: George B. Stefano, Melville, NY (US); Ji-Sheng Han, Beijing (CN)

Correspondence Address: FISH & RICHARDSON P.C., PO BOX 1022, MINNEAPOLIS, MN, 55440-1022, US

(73) Assignee: The Research Foundation of State University of New York, Albany, NY (US)

(21) Appl. No.: 11/673,969

(22) Filed: Feb. 12, 2007

(51) Int. Cl. A61N 1/00

(52) U.S. Cl. 607/46

(57) ABSTRACT

This document provides methods and materials for treating Raynaud's conditions such as Raynaud's syndrome, Raynaud's phenomenon, and Raynaud's disease. For example, this document provides methods for using electrical stimulation to reduce the severity of a symptom of a Raynaud's condition. Gloves and socks that can be used to reduce the severity of a symptom of a Raynaud's condition also are provided.

### BACKGROUND

1. Technical Field

This document relates to methods and materials involved in treating Raynaud's conditions such as Raynaud's syndrome, Raynaud's phenomenon, and Raynaud's disease.

2. Background Information

Raynaud's conditions (e.g., Raynaud's syndrome, Raynaud's phenomenon, and Raynaud's disease) are circulatory conditions characterized by exaggerated sensitivity to cold. This sensitivity can manifest itself in attacks that tend to be initiated by exposure to cold, sometimes even moderately cool temperatures. When an attack occurs, blood flow to the extremities (typically fingers, but also toes, ears, nose, and the tip of the tongue) can be restricted, leading to discomfort and numbness. The discomfort and numbness can keep sufferers from performing even the simplest of tasks, and in some cases, the attacks can cause significant nerve damage in the affected tissues.

### SUMMARY

This document provides methods and materials for treating Raynaud's conditions such as Raynaud's syndrome, Raynaud's phenomenon, and Raynaud's disease. For example, this document provides methods for using electrical current to reduce the severity of a symptom of a Raynaud's condition. This document also provides gloves and socks that can be used to reduce the severity of a symptom of a Raynaud's condition. The methods and materials provided herein can be used to reduce the discomfort and numbness associated with a Raynaud's condition. In addition, the methods and materials provided herein can allow mammals having a Raynaud's condition to lead more normal lives and can delay or prevent the onset of permanent damage from a Raynaud's condition.

In general, one aspect of this document features a glove comprising, or consisting essentially of, an electrical stimulation loop configured to deliver electrical stimulation to a portion of a human's hand when a human is wearing the glove on the human's hand, wherein the glove is configured to fit on the human's hand. The electrical stimulation loop can comprise first and second electrodes positioned in the glove such that electrical current travels from the first electrode through the portion of the human's hand and to the second electrode. The electrical stimulation loop can comprise a first electrode positioned in the glove to be facing a first side of the human's hand and a second electrode positioned in the glove to be a second side of the human's hand. The first side can be a palm side of the human's hand. The first side can be a back of the hand side of the human's hand. The second side can be a palm side of the human's hand. The second side can be a back of the hand side of the human's hand. The electrical stimulation loop can comprise first and second electrodes positioned in the glove to face opposite sides of the human's hand. The electrical stimulation loop can comprise an electrode positioned in the glove without substantial insulation between the electrode and the skin of the human's hand when the human is wearing the glove. The electrical stimulation loop can comprise first and second electrodes positioned in the glove to face opposite sides of a finger or thumb of the human's hand. The glove can comprise an electrical stimulation loop for each digit of the glove. The glove can comprise a control unit for controlling a parameter of the electrical stimulation. The parameter can be the frequency of the electrical stimulation, the voltage of the electrical stimulation, or the amperage of the electrical stimulation. The control unit can comprise an interface for receiving communication from a programming device. The glove can be configured to provide open finger and thumb tips.

In another aspect, this document features a sock comprising, or consisting essentially of, an electrical stimulation loop configured to deliver electrical stimulation to a portion of a human's foot when a human is wearing the sock on the human's foot, wherein the sock is configured to fit on the human's foot. The electrical stimulation loop can comprise first and second electrodes positioned in the sock such that electrical current travels from the first electrode through the portion of the human's foot and to the second electrode. The electrical stimulation loop can comprise a first electrode positioned in the sock to be facing a first side of the human's foot and a second electrode positioned in the sock to be a second side of the human's foot. The first side can be a top side of the human's foot. The first side can be a bottom side of the human's foot. The second side can be a top side of the human's foot. The second side can be a bottom side of the human's foot. The electrical stimulation loop can comprise first and second electrodes positioned in the sock to face opposite sides of the human's foot. The electrical stimulation loop can comprise an electrode positioned in the sock without substantial insulation between the electrode and the skin of the human's foot when the human is wearing the sock. The electrical stimulation loop can comprise first and second electrodes positioned in the sock to face opposite sides of a toe of the human's foot. The sock can comprise an electrical stimulation loop for each digit of the human's foot. The sock can comprise a control unit for controlling a parameter of the electrical stimulation. The parameter can be the frequency of the electrical stimulation, the voltage of the electrical stimulation, or the amperage of the electrical stimulation. The control unit can comprise an interface for receiving communication from a programming device.

In another aspect, this document features a method for treating a human having a Raynaud's condition. The method comprises, or consists essentially of, applying electrical stimulation to a hand or foot of the human under conditions wherein the discomfort or numbness of the Raynaud's condition is reduced. The discomfort or numbness of the Raynaud's condition can be reduced by more than 25 percent. The discomfort or numbness of the Raynaud's condition can be reduced by more than 50 percent. The frequency of the electrical stimulation can be between 1 and 200 hertz. The voltage of the electrical stimulation can be between 1 and 50 volts, and the amperage of the electrical stimulation can be between 1 and 50 milliamps. The method can comprise identifying the human as having the Raynaud's condition prior to applying the electrical stimulation. The method can comprise determining the severity of symptoms of the Raynaud's condition after applying the electrical stimulation. The method can comprise applying electrical stimulation to a hand of the human. The method can comprise applying electrical stimulation to a foot of the human. The method can comprise applying electrical stimulation for at least 30 minutes a day. The method can comprise applying electrical stimulation for at least one hour per day for more than five days.

In another aspect, this document features a method for treating a human having a Raynaud's condition. The method comprises, or consists essentially of, (a) putting a glove on a hand of the human, wherein the glove comprises an electrical stimulation loop configured to deliver electrical stimulation to a portion of the hand when the human is wearing the glove on the hand, wherein the glove is configured to fit on the hand, and (b) applying electrical stimulation to the hand of the human.

In another aspect, this document features a method for treating a human having a Raynaud's condition. The method comprises, or consisting essentially of, (a) putting a sock on a foot of the human, wherein the sock comprises an electrical stimulation loop configured to deliver electrical stimulation to a portion of the foot when the human is wearing the sock on the foot, wherein the sock is configured to fit on the foot, and (b) applying electrical stimulation to the foot of the human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a top view of a pair of gloves containing electrodes.

FIG. 2 is a cross sectional, side view of a single pair of electrodes that form an electrical stimulation loop for one finger of a glove.

FIG. 3 is a cross-sectional, side view of multiple pairs of electrodes that form multiple electrical stimulation loops for one finger of a glove.

FIG. 4 is a schematic diagram of a pair of gloves or socks.

FIG. 5 is a schematic diagram of a pair of gloves or socks.

FIG. 6 is a top view of a pair of gloves containing electrodes and having open finger tips.

FIG. 7 is a bar graph plotting the discomfort level for humans having a Raynaud's condition before and after treatment with electrical stimulation.

### DETAILED DESCRIPTION

This document provides methods and materials for treating Raynaud's conditions such as Raynaud's syndrome, Raynaud's phenomenon, and Raynaud's disease. For example, this document provides methods for using electrical stimulation to reduce the severity of a symptom (e.g., discomfort and numbness) of a Raynaud's condition. This document also provides gloves and socks that can be used to reduce the severity of a symptom of a Raynaud's condition.

As described herein, electrical stimulation can be applied to a user (e.g., a human user) to treat a Raynaud's condition. For example, a transcutaneous electrical nerve stimulation (TENS) unit can be used to deliver electrical stimulation to any body location of a human having a Raynaud's condition. Such body locations include, without limitation, a human's hands, feet, fingers, toes, wrists, ankles, ears, and nose. For example, a TENS unit can be used to apply electrical stimulation to any part of a human's hand such as particular fingers, the palm, thumb, or knuckle regions. When treating a human's hand, electrodes can be used to deliver electrical stimulation across the human's digits in any direction (e.g., from the top of a finger or thumb to the bottom of a finger of thumb, or vise versa). Any appropriate electrical stimulations parameters can be used. For example, a frequency of electricity between 1 and 200 hertz (e.g., between 1 and 150 hertz, between 1 and 100 hertz, between 1 and 50 hertz, between 1 and 25 hertz, between 2.5 and 150 hertz, between 5 and 150 hertz, between 10 and 150 hertz, between 5 and 25 hertz, or between 5 and 10 hertz) can be used, a voltage between 1 and 50 volts (e.g., between 1 and 40 volts, between 1 and 30 volts, between 1 and 20 volts, between 5 and 50 volts, between 10 and 50 volts, or between 2.5 and 25 volts) can be used, an amperage between 1 and 50 milliamps (e.g., between 1 and 40 milliamps, between 1 and 30 milliamps, between 1 and 20 milliamps, between 1 and 10 milliamps, between 2.5 and 50 milliamps, between 5 and 50 milliamps, between 2.5 and 25 milliamps, or between 5 and 25 milliamps) can be used, a pulse width between 100 and 2000 μsec (e.g., between 100 and 1500 μsec, between 100 and 1000 μsec, between 100 and 500 μsec, between 200 and 2000 μsec, between 500 and 2000 μsec, between 1000 and 2000 μsec, or between 500 and 1500 μsec) can be used, and a constant or pulsate output mode of operation can be used. In some cases, a frequency of 10 hertz, voltage of 50 volts, an amperage of up to 150 milliamps, a pulse width of 50 μsec can be used in a constant or pulsate output mode of operation.

In some cases, electrical stimulation can be applied for 10, 15, 20, 30, 45, 60, 90, 120, or more consecutive minutes. For example, electrical stimulation can be applied two hours per day. In some cases, electrical stimulation can be applied every day or every other day for any length of days (e.g., a week, month, or years). For example, electrical stimulation can be applied for between one to two hours at a time every day, every other day, every week, or every other week for a duration longer than a week (e.g., between a week to a year, or for a human's lifetime).

The methods and materials provided herein can be used to reduce the severity of a symptom (e.g., discomfort and numbness) of a Raynaud's condition. For example, the use of electrical stimulation as described herein can be used to reduce the severity of a symptom of a Raynaud's condition by, for example, at least 10, 20, 30, 40, 50, 60, 70, 80, 90, or more percent (e.g., between 10 and 90 percent; between 10 and 75 percent; between 10 and 50 percent; between 50 and 95 percent; between 60 and 95 percent; or between 75 and 95 percent). Any appropriate method can be used to determine the percent reduction. For example, patient questionnaires can be used to assess symptoms before and after treatment.

This document provides gloves (e.g., pairs of gloves) designed to apply electrical stimulation to the wearer's hands for the purpose of improving the symptoms of a Raynaud's condition. The user can wear the gloves and turn on electrical current when an electrical stimulation therapy is desired. A means for controlling the parameters of the electrical stimulation therapy can be included. In some cases, the provided gloves can be light weight and can lack of bulk. The gloves provided herein can be worn at almost all times, thus making treatment available to the user anytime symptoms are encountered, or anytime a preventative treatment is desired. In some cases, the gloves provided herein can be entirely self-contained, thereby allowing treatment to continue while the user goes about normal, daily activities.

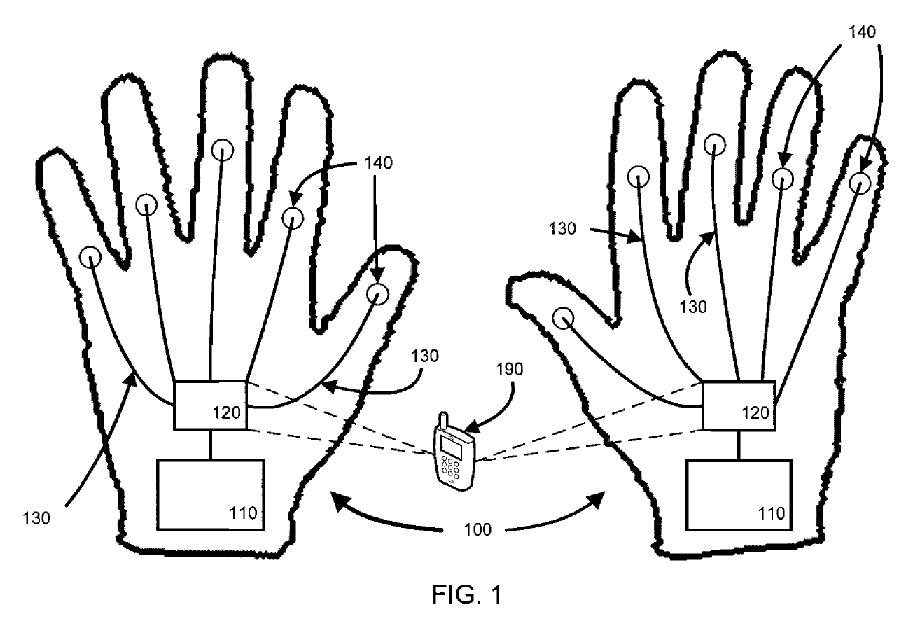

FIG. 1 is a top view diagram of a pair of gloves 100 that can provide electrical stimulation as a treatment for a Raynaud's condition. Gloves 100 can contain a power pack 110 for supplying power to a control unit 120. Power pack 110 can accept any power source such as a typical 9V battery that can be replaced when drained. In some cases, a rechargeable 9V battery can be used and recharged using a charger that is integrated into the gloves or is a separate device. In some cases, a rechargeable battery in a sealed power pack can be used. When charging is required, a user can plug a power cord directly into such a power pack. When plugged in, the device can also run on the AC power supplied through the cord. When on, control unit 120 can supply electrical stimulation through connection wires 130 to electrodes 140, both of which can be integrated into the glove material. In some cases, gloves 100 can have wires 130 and electrodes 140 that can be relocated based on a user's needs. In some cases, gloves 100 can be programmed using programming device 190. For example, programming device 190 can be used to set various parameters of the electrical stimulation (e.g., frequency, voltage, and amperage).

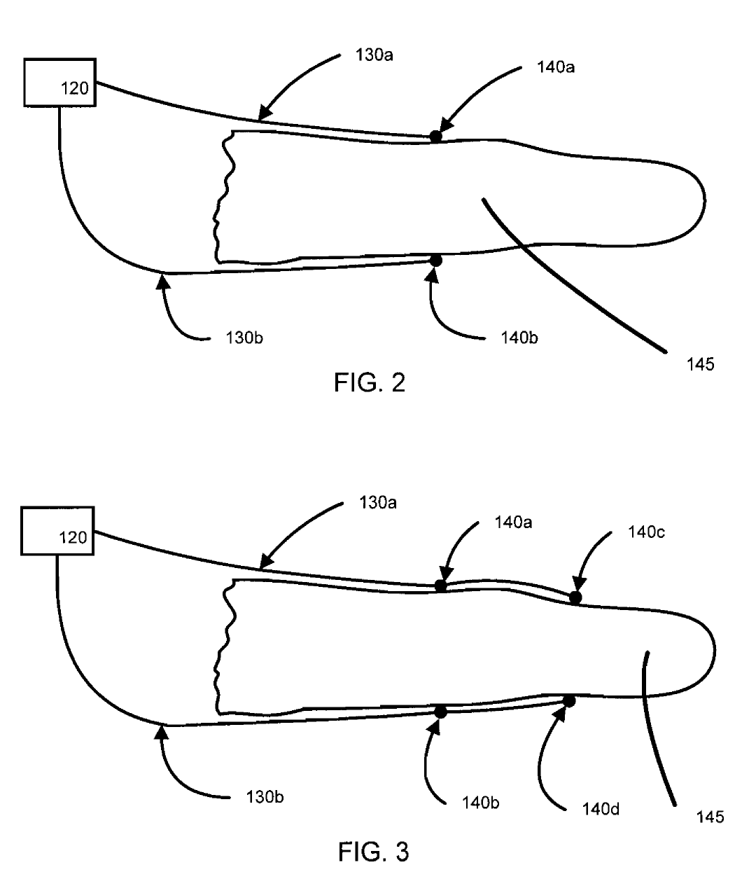

FIG. 2 is a schematic diagram of an electrical stimulation loop that can be present in a glove. Electrical stimulation loop can include a pair of wires 130 and electrodes 140. In some cases, current can travel down connection wire 130a to electrode 140a, where it can enter digit 145 (e.g., a finger or thumb). The current can pass through digit 145, into electrode 140b, through wire 140b, and back to control unit 120.

Any number of electrical stimulation loops can be incorporated into gloves 100. In some cases, each of electrode pairs (140a, 140b) can be activated simultaneously or serially in any combination. For example, a user can program gloves 100 to deliver electrical stimulation to the user's first three digits simultaneously in a frequent manner and to the user's other two digits in secession in a less frequent manner. With reference to FIG. 3, electrodes 140a, 140b, 140c, and 140d can be arranged into zones that allow multiple electrode pairs (140a, 140b; and 140c, 140d) to be activated using a single pair of wires 130a, 130b. The electrodes of a glove provided herein can be any appropriate shape (e.g., a circular, square, rectangle, or oval).

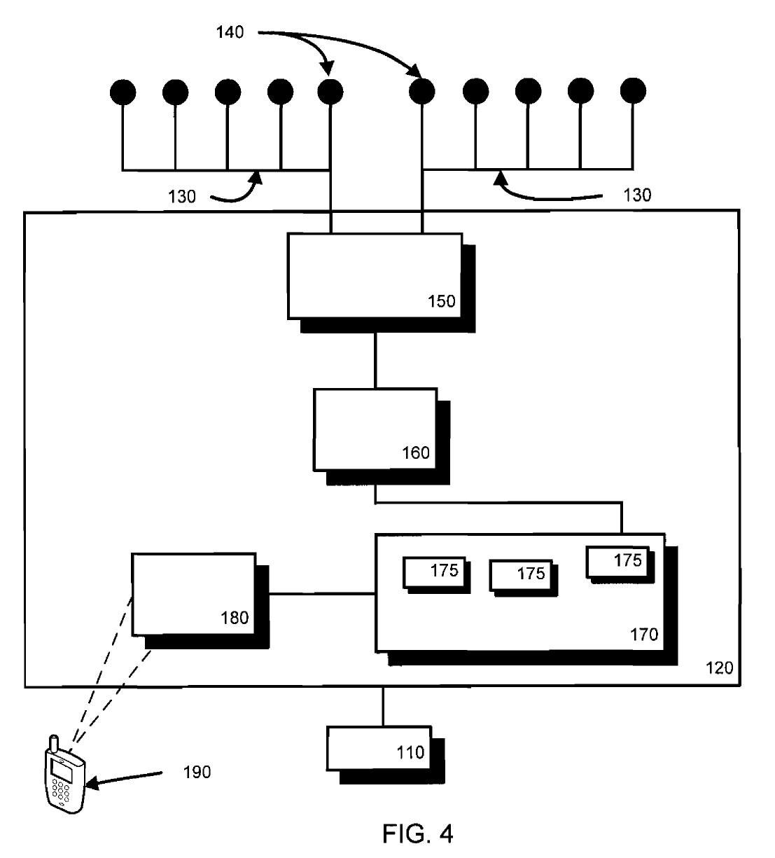

FIG. 4 is a schematic view of one embodiment of gloves 100. A single waveform can be sent simultaneously to five different pairs of electrodes 140 via wires 130. In this example, one electrode pair can be positioned to provide electrical stimulation to each digit of a user's hand. Control unit 120 can contain multiple sub-units that can be involved in the creation and control of an electrical stimulation therapy. Transformer 150 can be used to supply a controlled voltage to electrodes 140 via wires 130, and pulse generator 160 can be used to create a wave form that can be supplied to transformer 150. CPU 170 can be contained within control unit 120 and can be used to store a therapeutic program and initiate electrical stimulation.

Multiple parameters of an electrical stimulation can be adjusted, thus maximizing the benefit for a user. In some cases, control unit 120 can contain knobs or switches to allow a user to control the frequency, voltage, and amperage. In some cases, CPU 170 can store and allow for adjustment of many electrical stimulation parameters 175. Examples of such parameters that can be adjustable include, without limitation, frequency (Hz), voltage intensity (V), pulse amplitude (mA), pulse width (μsec), and mode of operation. A conventional mode creates a simple sign or square wave. A burst mode can be used to supply a series of bursts at a pre-set frequency. Each burst can contain a number of pulses with their own frequency and pulse width. In this case, a series of short, equally spaced pulses can be applied with a pause between bursts of pulses. Other, more complex modes currently available in TENS units, such as interference or pre-modulated, can be used.

Control unit 120 can contain one or more knobs, switches, or buttons for controlling any electrical stimulation parameter 175. In some cases, control unit 120 can contain interface 180. Interface 180 can be located within control unit 120 and can receive communication from an external programming device 190, which can be used for programming electrical stimulation parameters 175. In some cases, interface 180 can be a USB interface that a user can connect to a computer via a USB cable. The computer can then be used to configure any electrical stimulation parameter 175 or select one or more pre-configured parameter sets. In some cases, interface 180 is a Bluetooth® device used to communicate with programming device 190, which can be a Bluetooth® enabled PDA containing appropriate programming software. Interface 180 can be designed to connect with virtually any device that can be used to program the gloves via any connection, both wired and wireless.

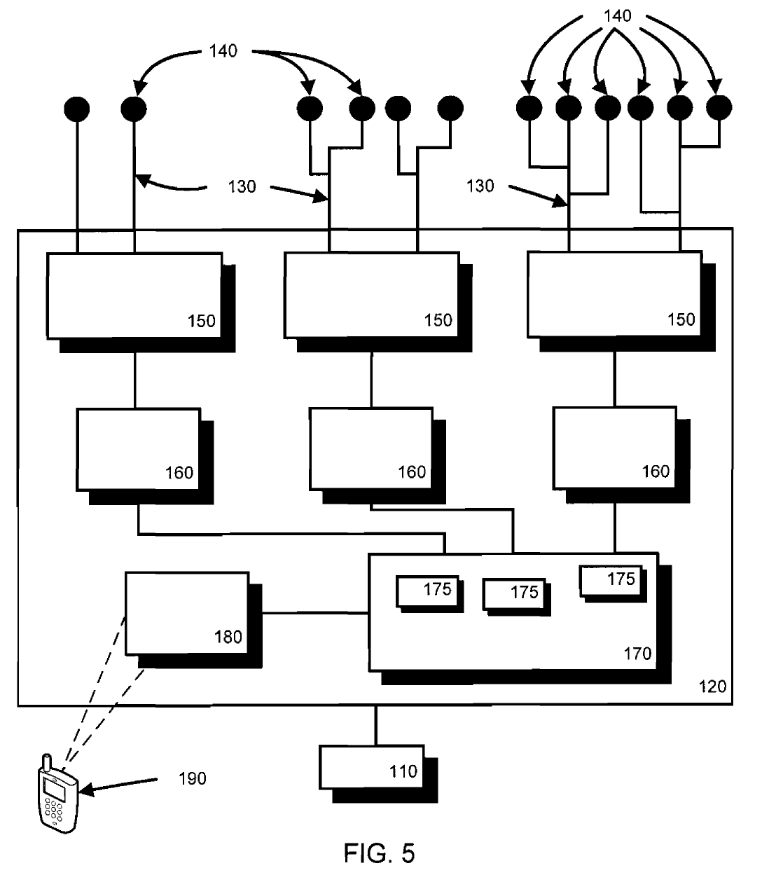

With reference to FIG. 5, gloves can be configured to use multiple pulse generators 160 and transformers 150. In such cases, a therapeutic program can initiate separate waveforms in separate wave generators 160. These wave forms can then be transformed to appropriate voltages in transformers 150 and allowed to pass through wires 130 to electrodes 140. Additional embodiments can contain any number of transformers 150), pulse generators 160, and CPUs 170.

The gloves provided herein can each contain a control unit 120. In some cases, a single control unit can be used to control both gloves of a pair. Transformer 150, pulse generator 160, CPU 170, and interface 180 can be located inside control unit 120, or one or more of these sub-units can exist outside control unit 120. For example, power pack 110 can be configured into control unit 120 or can be configured to exist outside of control unit 120.

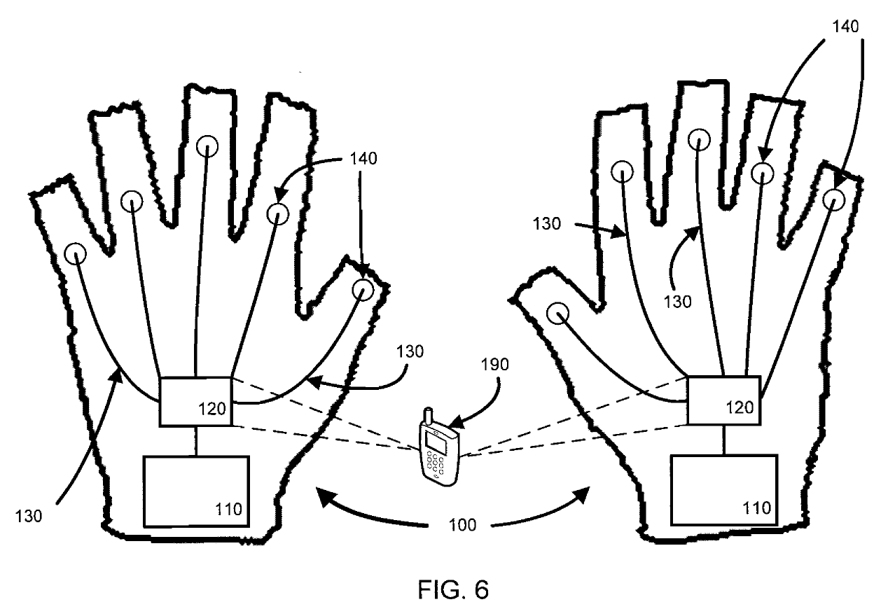

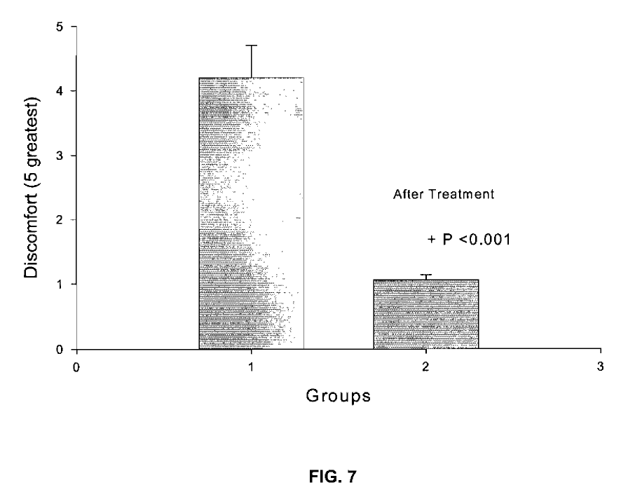

With reference to FIG. 6, gloves 100 can be designed such that the finger tips are removed, allowing a user to perform more precise tasks.

This document also provides other devices (e.g., socks, ear muffs, and patches that can be placed over a user's nose) that can be designed to contain components similar to those described herein for gloves such that a user can deliver electrical stimulation to other body parts. For example, socks containing multiple electrical stimulation loops can be designed to deliver electrical stimulation to a human's feet.

The invention will be further described in the following examples, which do not limit the scope of the invention described in the claims.

### EXAMPLES

### Example 1

### Treating Raynaud's Conditions

Five humans diagnosed as having Raynaud's phenomenon were instructed individually on the operation of a TENS unit (HANS model LH 202; Beijing HuaWei Company, Beijing, China). Each human also was asked to complete the pre-treatment portions of a questionnaire containing questions about the person's condition and discomfort and numbness levels. The discomfort and numbness levels were scored on a scale from one to five with five being the most severe. Each human was asked to use the TENS unit as follows for two to three hours everyday for seven days. The humans were instructed to (a) locate discomforted and/or numb fingers, (b) place two of the TENS unit adhesive pads directly over the located areas, (c) turn the TENS unit on and set the desired frequency to 10 hertz, (d) increase the intensity until a sensation is perceived in the desired fingers, (e) decrease the intensity to a level of minimal sensation, (f) continue to apply the electrical stimulation from the TENS unit at the adjusted setting for at least 30 minutes up to two hours, (g) turn TENS unit off and remove the TENS unit adhesive pads, and (h) record the response on the questionnaire at the end of each day.

Treatment with the TENS unit resulted in a statistically significant improvement in the vascular flow as reported by less discomfort and numbness (FIG. 1). In addition, all five humans diagnosed with Raynaud's phenomenon reported an overall improvement. The fingers appeared to be pinker after the seven-day treatment period, indicating improved vascular flow. These results indicate that electrical stimulation can be used to treat Raynaud's phenomenon.

### Other Embodiments

It is to be understood that while the invention has been described in conjunction with the detailed description thereof the foregoing description is intended to illustrate and not limit the scope of the invention, which is defined by the scope of the appended claims. Other aspects, advantages, and modifications are within the scope of the following claims.

What is claimed is:

1. A glove comprising an electrical stimulation loop configured to deliver electrical stimulation to a portion of a human's hand when a human is wearing said glove on said human's hand, wherein said glove is configured to fit on said human's hand.

2. The glove of claim 1, wherein said electrical stimulation loop comprises first and second electrodes positioned in said glove such that electrical current travels from said first electrode through said portion of said human's hand and to said second electrode.

3. The glove of claim 1, wherein said electrical stimulation loop comprises a first electrode positioned in said glove to be facing a first side of said human's hand and a second electrode positioned in said glove to be a second side of said human's hand.

4. The glove of claim 3, wherein said first side is a palm side of said human's hand.

5. The glove of claim 3, wherein said first side is a back of the hand side of said human's hand.

6. The glove of claim 3, wherein said second side is a palm side of said human's hand.

7. The glove of claim 3, wherein said second side is a back of the hand side of said human's hand.

8. The glove of claim 1, wherein said electrical stimulation loop comprises first and second electrodes positioned in said glove to face opposite sides of said human's hand.

9. The glove of claim 1, wherein said electrical stimulation loop comprises an electrode positioned in said glove without substantial insulation between said electrode and the skin of said human's hand when said human is wearing said glove.

10. The glove of claim 1, wherein said electrical stimulation loop comprises first and second electrodes positioned in said glove to face opposite sides of a finger or thumb of said human's hand.

11. The glove of claim 1, wherein said glove comprises an electrical stimulation loop for each digit of said glove.

12. The glove of claim 1, wherein said glove comprises a control unit for controlling a parameter of said electrical stimulation.

13. The glove of claim 12, wherein said parameter is the frequency of said electrical stimulation, the voltage of said electrical stimulation, or the amperage of said electrical stimulation.

14. The glove of claim 12, wherein said control unit comprises an interface for receiving communication from a programming device.

15. The glove of claim 1, wherein said glove is configured to provide open finger and thumb tips.

16. A method for treating a human having a Raynaud's condition, wherein said method comprises applying electrical stimulation to a hand or foot of said human under conditions wherein the discomfort or numbness of said Raynaud's condition is reduced.

17. The method of claim 16, wherein the discomfort or numbness of said Raynaud's condition is reduced by more than 25 percent.

18. The method of claim 16, wherein the discomfort or numbness of said Raynaud's condition is reduced by more than 50 percent.

19. The method of claim 16, wherein the frequency of said electrical stimulation is between 1 and 200 hertz.

20. The method of claim 16, wherein the voltage of said electrical stimulation is between 1 and 50 volts, and the amperage of said electrical stimulation is between 1 and 50 milliamps.

21. The method of claim 16, wherein said method comprises identifying said human as having said Raynaud's condition prior to applying said electrical stimulation.

22. The method of claim 16, wherein said method comprises determining the severity of symptoms of said Raynaud's condition after applying said electrical stimulation.

23. The method of claim 16, wherein said method comprises applying electrical stimulation to a hand of said human.

24. The method of claim 16, wherein said method comprises applying electrical stimulation to a foot of said human.

25. The method of claim 16, wherein said method comprises applying electrical stimulation for at least 30 minutes a day.

26. The method of claim 16, wherein said method comprises applying electrical stimulation for at least one hour per day for more than five days.

27. A method for treating a human having a Raynaud's condition, wherein said method comprises:
(a) putting a glove on a hand of said human, wherein said glove comprises an electrical stimulation loop configured to deliver electrical stimulation to a portion of said hand when said human is wearing said glove on said hand, wherein said glove is configured to fit on said hand, and
(b) applying electrical stimulation to said hand of said human.
